(19) United States
(12) Patent Application Publication
(10) Pub. No.: US 2011/0144452 A1
(43) Pub. Date: Jun. 16, 2011

(54) APPARATUS AND METHOD FOR DETERMINING EMOTIONAL QUOTIENT ACCORDING TO EMOTION VARIATION

(76) Inventors: Hyun-Soon SHIN, Daejeon (KR); Jun Jo, Daejeon (KR); Yong-Kwi Lee, Seoul (KR); Jun-Sik Choi, Daejeon (KR); Jin-Hoon Kim, Daejeon (KR)

(21) Appl. No.: 12/908,589

(22) Filed: Oct. 20, 2010

(30) Foreign Application Priority Data
Dec. 10, 2009 (KR) 10-2009-0122658

(51) Int. Cl. A61B 5/00

(52) U.S. Cl. 600/300

(57) ABSTRACT

The present invention relates to an apparatus and a method for determining emotional quotient according to emotional variation. The method for determining emotional quotient according to emotional variation includes sensing bio signals and environmental signals from a plurality of sensors; defining an emotional variation threshold value for each signal sensed at the sensing; extracting the emotional signal by analyzing the bio signals and inferring the emotional quotient for each bio signal based on the extracted emotional signal and the environmental signals; determining the emotional variation threshold values and the emotional quotient based on the inference results of the emotional quotient; and evaluating the determined emotional quotient. With the present invention, it can provide the emotional care service capable of informing and communicating the true emotional state of the person to user.

### CROSS REFERENCE TO RELATED APPLICATIONS

This application claims priority to Korean Patent Application No. 10-2009-0122658 filed on Dec. 10, 2009, the entire contents of which are herein incorporated by reference.

### BACKGROUND OF THE INVENTION

1. Field of the Invention

The present invention relates to an apparatus and a method for determining emotional quotient according to emotional variation, and more particularly, to an apparatus and a method for setting personalized emotional variation threshold and determining personalized emotional quotient from a re-inference algorithm by performing feedback-based self diagnosis and service-based on monitoring statistical-based multimodal emotional variation threshold values for emotional variation according to the physiology and characteristic of individual users.

2. Description of the Related Art

Recently, a need for a recognition and awareness technology as a method for intelligently determining and providing a user's intentions and services in consideration of emotion and person's recognition aspect has been on the rise.

By the need, many researches have been conducted. However, the emotion information services are not provided by the emotion recognition technology.

A future society that is a personalizing society and an aging society needs an emotion based service that can communicate emotion such as pleasure.

Therefore, a need exists for a method for setting personalized emotional variation threshold and determining personalized emotional quotient capable of recognizing emotion meeting a person by generating emotional variation threshold data for determining the physiology and physical characteristic of the person, the current environment, and the personalized emotional quotient meeting the condition in determining the statistical based common (average) rule base based on the emotional quotient.

### SUMMARY OF THE INVENTION

It is an object of the present invention to provide a method for recognizing emotion by accurately inferring the emotional variation according to the physiology and characteristics of the individual user.

It is another object of the present invention to provide optimal emotional services meeting characteristics of a person based on personal emotion by providing method for setting personalized emotional variation threshold and determining personalized emotional quotient from a re-inference algorithm by performing feedback-based self diagnosis and service-based on monitoring on statistical-based multimodal emotional variation threshold values.

According to an exemplary embodiment of the present invention, there is provided a method for determining emotional quotient according to emotional variation, including: defining an emotional variation threshold value based on bio signals and environmental signals sensed from a plurality of sensors; extracting emotional signal by analyzing the bio signals and inferring the emotional quotient for each bio signal based on the extracted emotional signal and the environmental signals; determining the emotional variation threshold values and the emotional quotient based on the inference results of the emotional quotient; evaluating the determined emotional quotient; and re-determining the emotional variation threshold value and the emotional quotient of the determining according to the evaluation results of the evaluating.

The determining the emotional variation threshold values and the emotional quotient includes performing multimodal emotional inference by using the emotional quotient for each bio signal as the multi input, the emotional variation threshold value and the emotional quotient being determined according to the multimodal emotional inference results.

The evaluating includes determining a way for evaluating the emotional quotient for evaluating the emotional quotient among self diagnosis and emotional care service feedback.

The evaluating further includes querying whether the user agrees on the recognized emotional state when self diagnosis is used.

The re-determining includes if agreed on the recognized emotional state by the response to the query, fixing the emotional variation threshold values in an emotional threshold management table, and if not, adjusting the level of the information sensed from the bio signals and the environmental signals to correct the emotional variation threshold value.

The evaluating further includes analyzing service consuming patterns according to the user's emotional state to select the emotional care service meeting the emotional quotient when the emotional care service feedback is used; monitoring the emotional variation while the selected emotional care service is provided; and analyzing the emotional variation in the current emotional quotient according to the monitored results.

The re-determining further includes: when the current emotion of the user is not varied, adjusting the level of the emotional quotient if it is determined that it is negative emotion by searching the emotional quotient, and if not, fixing the emotional variation threshold values in the emotional threshold management table.

The re-determining includes when the current emotion of the user is varied, confirming whether the emotion is varied to the positive emotion; and when the emotion is varied to the positive emotion, fixing the emotional variation threshold values in the emotional threshold management table.

The method for determining emotional quotient further includes the current emotion of the user is varied, confirming whether the emotion is varied to the negative emotion; and when the emotion is varied to the negative emotion, adjusting the level of the emotional quotient.

According to another embodiment of the present invention, there is provided an apparatus for determining emotional quotient according to emotional variation, including: a plurality of sensors that sense bio signals and environmental signals; a sensing signal processor that processes sensing signals sensed by the plurality of sensors; and an emotional signal processor that defines emotional variation threshold values as a reference capable of recognizing emotional variation, analyzes the sensing signals from the sensing signal processor to extract emotional signal, and infers the emotional quotient based on the extracted emotional signal and the environment signals.

The emotional signal processor further includes: an error condition recognizing unit that analyzes the environmental information to sense the error condition of the extracted emotional signal; and an emotional signal compensator that compensates the error condition recognized by the error condition recognizing unit to optimize the emotional signal.

The emotional signal compensator adjusts the level of the emotional quotient to compensate for the error condition of the emotional signal.

The emotional signal processor extracts the emotional signal from the bio signals based on the emotional variation threshold values.

The emotional signal processor further includes an emotional signal information managing unit that manages the emotional variation threshold values, the emotional quotient, the emotional signal, and the state information of the emotional signal feedback from the corresponding user.

The emotional signal information managing unit evaluates the emotional quotient based on the feedback result from the user and determines the optimized emotional variation threshold values and the emotional quotient according to the evaluation results.

The emotional signal information managing unit evaluates the emotional quotient through self diagnosis or the emotional care service feedback for the emotional state recognition result.

The emotional signal information managing unit queries whether the user agrees on the recognized emotional state when the emotional quotient is evaluated through self diagnosis, to evaluate the emotional quient according to the response from the user.

The emotional signal information managing unit monitors the emotional variation while the emotional care service is provided when the emotional quotient is evaluated through the emotional care service feedback, to confirm whether the emotion for the recognized emotional state is varied and to evaluate the emotional quotient according to the result.

According to exemplary embodiments of the present invention, it provides the method for recognizing emotion by accurately inferring the emotional variation according to the physiology and characteristics of the individual user, thereby making it possible to provide the emotional service meeting each person's characteristics.

Further, the present invention can provide the optimal emotional services meeting the characteristics of the person based on the personal emotion by providing the method for setting the personalized emotional variation threshold and determining the personalized emotional quotient from the re-inference algorithm by performing the feedback-based self diagnosis and the service-based on monitoring the statistical-based multimodal emotional variation threshold values.

### BRIEF DESCRIPTION OF THE DRAWINGS

FIG. 1 is a block diagram for explaining a configuration of an apparatus for determining emotional quotient according to emotional variation of the present invention;

FIG. 2 is a block diagram for explaining a detailed configuration of a sensing signal processor according to the present invention;

FIG. 3 is a block diagram for explaining a detailed configuration of a sensing signal processor according to the present invention;

FIG. 4 is a block diagram for explaining a detailed configuration of an emotional signal communicating unit according to the present invention;

FIG. 5 is a block diagram for explaining a detailed configuration of a device controller according to the present invention;

FIG. 6 is a flowchart showing an operational flow of a method for determining emotional quotient according to the emotional variation of the present invention; and

FIGS. 7 to 10 are flow charts showing an operational flow of the method for evaluating the determined emotional quotient.

### DETAILED DESCRIPTION OF THE PREFERRED EMBODIMENTS

The present invention relates to an apparatus for determining emotional quotient according to emotion variation, and in particular to an apparatus for extracting an emotional signal from a bio signal by being applied to the sensing based emotional service apparatus.

Hereinafter, exemplary embodiments of the present invention will be described with reference to the accompanying drawings.

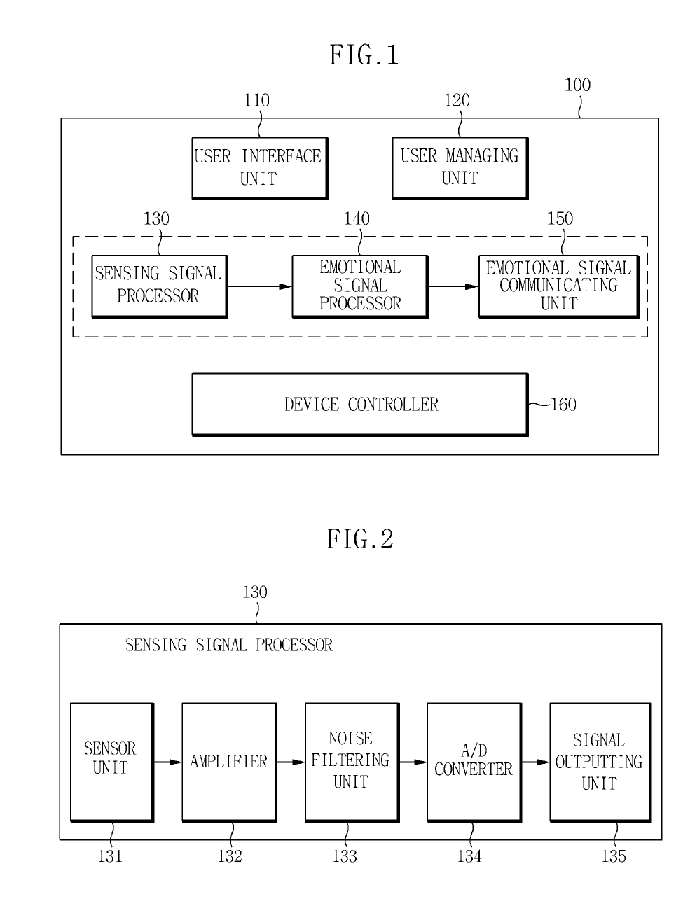

FIG. 1 is a block diagram for explaining a configuration of an apparatus for determining an emotional quotient according to emotional variation of the present invention.

Referring to FIG. 1, an apparatus 100 for determining emotional quotient according to emotional variation of the present invention includes a user interface unit 110, a user managing unit 120, a sensing signal processor 130, an emotional signal processor 140, an emotional signal communication unit 150, and a device controller 160.

The user interface unit 110 receives a unit that receives instructions from the user or outputs a predetermined signal to a user.

The user managing unit 120 performs the authentication and registration of the accessing user through the user interface unit 110 and manages the corresponding user.

The sensing signal processor 130 uses a plurality of sensors to sense bio signals and environmental signals. The detailed configuration of the sensing signal processor 130 will be described with reference to FIG. 2.

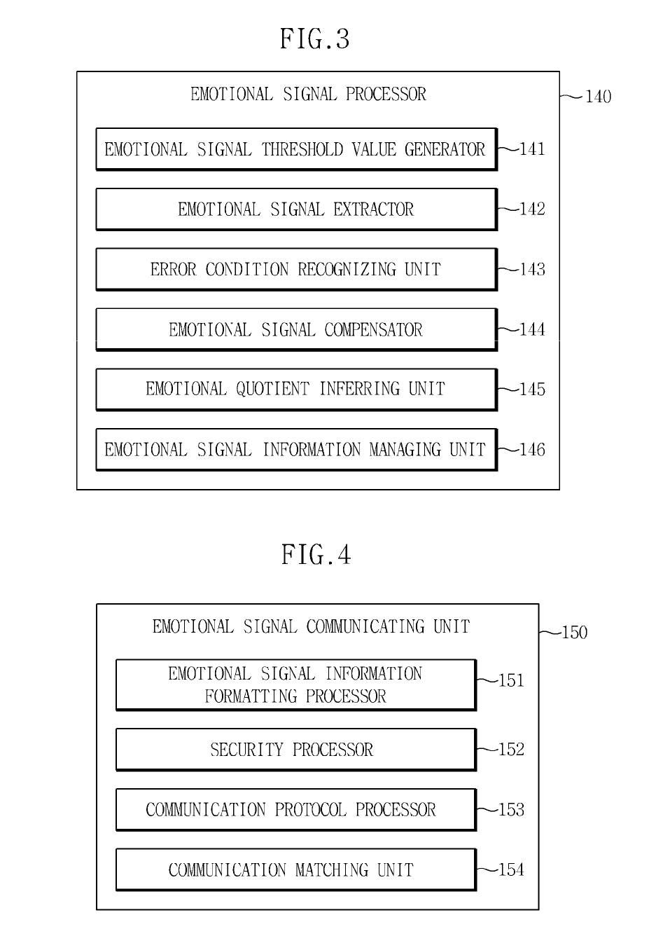

The emotional signal processor 140 extracts and processes the emotional signal by analyzing and processing the sensed signals by the sensing signal processor 130. The detailed configuration of the emotional signal processor 140 will be described with reference to FIG. 3.

The emotional signal communication unit 150 transmits the emotional signal generated by the emotional signal processor 140 to the peripheral terminal apparatuses and receives a response from the peripheral terminal apparatuses. The detailed configuration of the emotional signal communication unit 150 will be described with reference to FIG. 4.

The device controller 160 controls an operation of hardware and software of the apparatus for determining emotional quotient according to the present invention.

FIG. 2 is a block diagram for explaining a detailed configuration of a sensing signal processor according to the present invention.

As shown in FIG. 2, the sensing signal processor 130 includes a sensor unit 131, an amplifier 132, a noise filtering unit 133, an A/D converter 134, and a signal outputting unit 135.

First, the sensor unit 131 is a unit that includes a plurality of sensors to sense multimodal signals. In this case, the sensor unit 131 may include a PPG sensor, a GSR sensor, a temperature sensor, an accelerator sensor, an audio sensor, an infrared sensor, etc., that perform the unconstrained and unaware senses. Therefore, the sensor unit 131 senses the bio signals and the environmental signals through the plurality of sensors.

The amplifier 132 serves to amplify the signals sensed by the plurality of sensors included in the sensor unit 131.

The noise filtering unit 133 serves to remove the external noise from the signals amplified by the amplifier 132.

The A/D converter 134 serves to convert an analog signal into a digital signal and the signal outputting unit 135 outputs the bio signals into digital signal form converted by the A/D converter 134 to the emotional signal processor 140.

Meanwhile, FIG. 3 is a block diagram for explaining a detailed configuration of a sensing signal processor according to the present invention.

As shown in FIG. 3, the emotional signal processor 140 is configured to include an emotional signal threshold generator 141, an emotional signal extractor 142, an error condition recognizing unit 143, an emotional signal compensator 144, an emotional quotient inferring unit 145, and an emotional signal information managing unit 146.

The emotional signal threshold generator 141 generates an emotional variation threshold value that can recognize the emotional variation. In addition, the threshold signal threshold generator 141 controls the generated emotional variation threshold value.

The emotional signal extractor 142 extracts the emotional signal from the bio signals sensed by the sensing signal processor 130 based on the emotional variation threshold value generated by the emotional signal generator 141.

The error condition recognizing unit 143 analyzes the condition/environment information to sense the error condition of the emotional signal extracted by the emotional signal extractor 142.

The emotional signal compensator 144 compensates the error condition of the emotional signal recognized by the error condition recognizing unit 143 to optimize the corresponding emotional signal.

The emotional signal inferring unit 145 is a unit that performs the emotional inference by using the emotional signal and the condition signals as inputs. In this case, the emotional quotient inferring unit 145 uses the emotional quotient for each sensing signal from each sensor as multi inputs to perform the multimodal emotional inference, thereby inferring and determining the emotional signal variation threshold value and the emotional quotient.

The emotional signal information managing unit 146 manages the emotional signal extracted from the bio signals and the emotional signal state information feedback from the user, etc.

FIG. 4 is a block diagram for explaining a detailed configuration of an emotional signal communication unit according to the present invention.

As shown in FIG. 4, the emotional signal communication unit 150 includes an emotional signal information formatting processor 151, a security processor 152, a communication protocol processor 153, and an emotional signal communication matching unit 154.

The emotional signal information formatting processor 151 formats the emotional signal information for communicating the emotional signal in order to provide the emotional signal to the peripheral devices and terminals.

The security processor 152 performs the security function in order to secure the privacy of the personal emotional signal information.

The communication protocol processor 153 performs the operation for communicating the emotional signal. In addition, the communication matching unit 154 performs the signal matching for communicating wireless emotional signal.

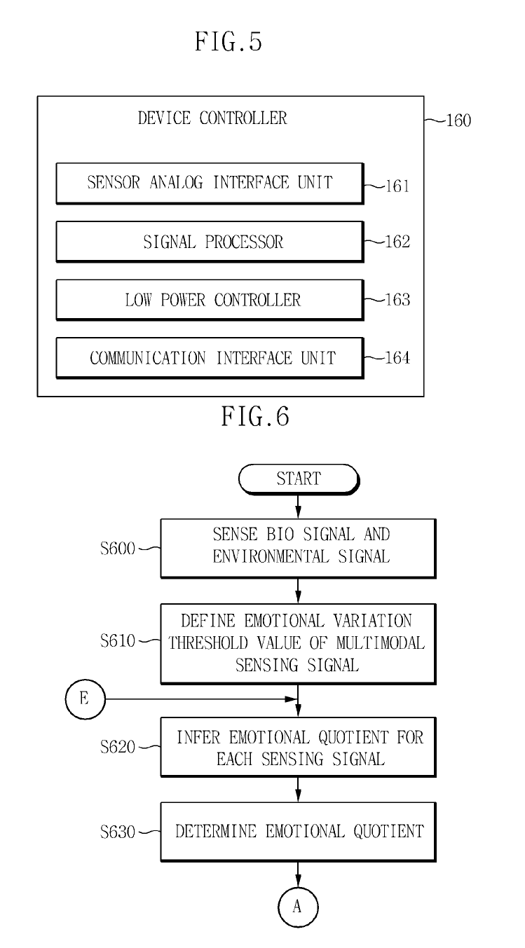

FIG. 5 is a block diagram for explaining a detailed configuration of a device controller according to the present invention.

As shown in FIG. 5, the device controller 160 includes a sensor analog interface unit 161, a signal processor 162, a low power controller 163, and a communication interface unit 164.

The sensor analog interface unit 161 receives the analog signals from a plurality of sensors. The signal processor 162 controls the execution of the booting and SW module execution of the apparatus for determining the emotional quotient according to the present invention. The low power controller 163 provides low power service in the apparatus for determining the emotional quotient according to the present invention. The communication interface unit 164 performs the emotional signal wireless communication.

The operation of the present invention configured as described above will be described in more detail.

FIG. 6 is a flowchart showing an operational flow of a method for determining the emotional quotient according to the emotional variation of the present invention.

As shown in FIG. 6, the sensing signal processor 130 senses the bio signals and the environmental signal from the signals sensed by the plurality of sensors (S600).

The emotional signal processor 140 receives the personal emotional quotient request by using the bio signals and the environmental signals sensed by the sensing signal processor 130 as inputs, it defines a statistical-based rule base emotional variation threshold value for each of the multimodal sensing signals (S610) and infers the emotional quotient for each sensing signal (S620).

In this case, the emotional signal processor 140 determines the emotional quotient based on the results of step ‘S620’ (S630).

Step ‘S620’ is step for determining the emotional quotient primary, and step ‘S630’ is step for confirming result of the determining the emotional quotient primary.

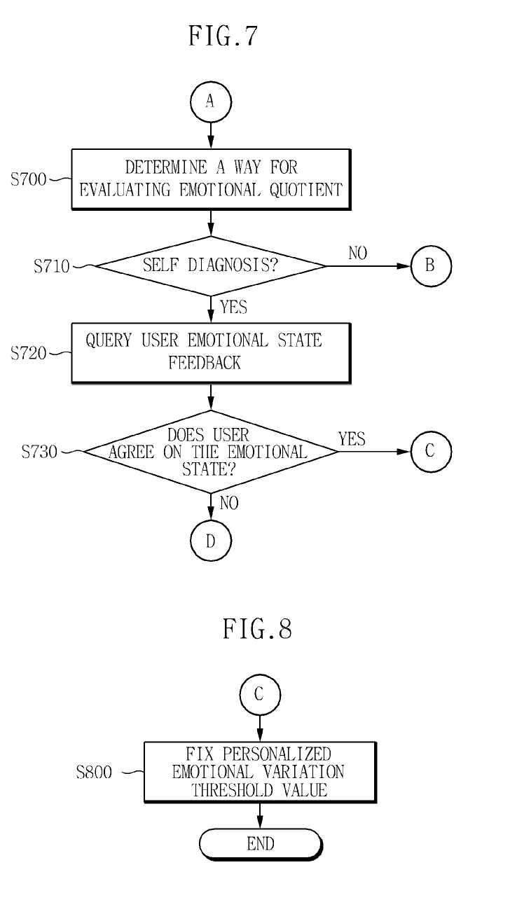

Thereafter, the emotional signal information managing unit 146 performs steps after ‘A’ of FIG. 7 in order to evaluate the determined emotional quotient.

FIGS. 7 to 10 are a flow chart showing an operational flow of the method for evaluating the emotional quotient determined in FIG. 6.

Referring to FIG. 7, the emotional signal information managing unit 146 determines a way for evaluating the emotional quotient by evaluating whether the definition of the emotional quotient is optimized after step ‘S630’ (S700).

If the evaluation way determined in step ‘S700’ is the self diagnosis (S710), it feedbacks the recognized emotional state to the user to query whether the feedback emotional state agrees with the corresponding emotional state (S720).

In this case, when the user agrees on the recognized emotional state (S730), it performs steps after ‘C’ of FIG. 8. In other words, the emotional signal information managing unit 146 fixes the emotional variation threshold value used in the current emotional recognition in the emotional threshold management table (S800) and ends the emotional quotient determining operation.

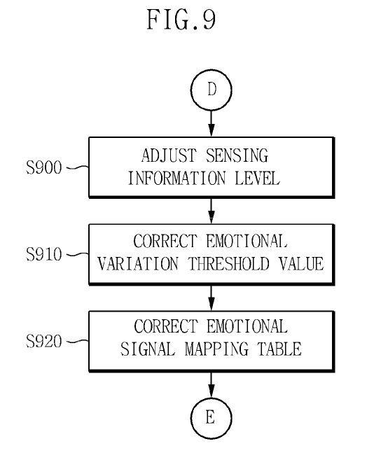

On the other hand, when the user does not agree on the recognized emotional state from the response (S730), the emotional signal threshold value generator 141 performs steps after ‘D’ of FIG. 9. In other words, the emotional signal threshold value generator 141 adjusts a level such as the time information, the environmental information, the condition information, the bio information, etc. from the sensing signal processor 130 (S900) and corrects the threshold value by performing the emotional variation threshold reproducing algorithm (S910).

Thereafter, the emotional signal information managing unit 146 corrects the emotional signal mapping table for each emotional signal threshold value (S920) and returns to ‘E’ of FIG. 6 and performs steps after step ‘S620’.

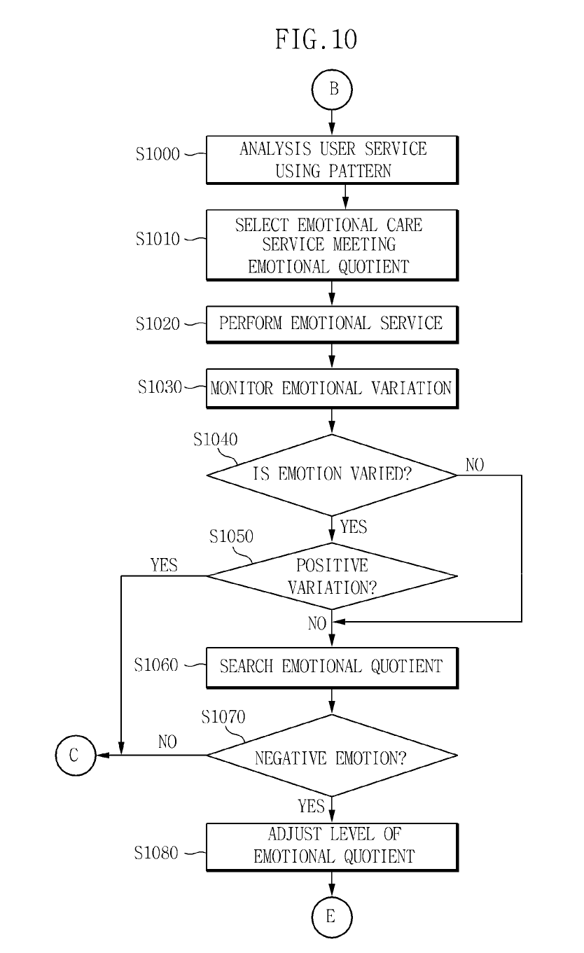

Meanwhile, the evaluation method determined in step ‘S700’ is not self diagnosis (S710), then steps after ‘B’ of FIG. 10 are performed.

As shown in FIG. 10, the emotional signal information managing unit 146 analyzes the user service using pattern for evaluating the emotional quotient (S1000) and selects the emotional care service meeting the emotional quotient according to the analyzed results (S1010).

Thereafter, the emotional signal information managing unit 146 performs the emotional care service selected in step ‘S1010’ (S1020) and monitors the emotional variation for a predetermined time to search the variation in the emotional state (S1030).

If there is no emotional variation (S1040), the emotional signal information managing unit 146 searches the current emotional quotient (S1060). In this case, when the current emotion of the user is the negative emotion (S1070), the emotional signal information managing unit 146 adjusts the level of the emotional quotient (S1080) to perform steps after ‘A’ of FIG. 7.

On the other hand, when the current user's emotion is not the negative emotion (S1070), the emotional signal information managing unit 146 performs steps after ‘C’ of FIG. 8. In other words, if there is no emotional variation in the positive emotion or the peaceful emotion, the emotional signal information managing unit 146 fixes the emotional variation threshold values used to recognize the current emotion to the personal emotional threshold management table and ends the emotional quotient recognition processing.

Meanwhile, when there is no emotional variation (S1040), the emotional signal information managing unit 146 confirms whether the emotional variation is the positive variation (S1050). If it is determined that the emotional variation is the positive variation, the emotional signal information managing unit 146 performs steps after ‘C’ of FIG. 8 to fix the emotion variation threshold values used to recognize the current emotion in the personal emotion threshold management table and end the emotional quotient recognition processing.

If it is determined that the emotional variation is not the positive variation (S1050), that is, the negative variation, the emotional signal information managing unit 146 searches the emotional quotient (S1060) to adjust the level of the emotional quotient (S1080) when the emotion of the user is the negative emotion (S1070) and performs steps after ‘A’ of FIG. 7.

As described above, although the apparatus and method for determining emotional quotient according to emotional variation of the present invention is described with reference to the illustrated drawings, the present invention is not limited to the embodiments disclosed in the specification and the drawings but can be applied within the technical scope of the present invention.

What is claimed is:

1. A method for determining emotional quotient according to emotional variation, comprising:
defining an emotional variation threshold value based on bio signals and environmental signals sensed from a plurality of sensors;
extracting emotional signal by analyzing the bio signals and inferring the emotional quotient for each bio signal based on the extracted emotional signal and the environmental signals;
determining the emotional variation threshold values and the emotional quotient based on the inference results of the emotional quotient;
evaluating the determined emotional quotient; and
re-determining the emotional variation threshold value and the emotional quotient of the determining according to the evaluation results of the evaluating.

2. The method for determining emotional quotient of claim 1, wherein the determining the emotional variation threshold values and the emotional quotient includes performing multimodal emotional inference by using the emotional quotient for each bio signal as the multi input, the emotional variation threshold value and the emotional quotient being determined according to the multimodal emotional inference results.

3. The method for determining emotional quotient of claim 1, wherein the evaluating includes determining a way for evaluating the emotional quotient for evaluating the emotional quotient among self diagnosis and emotional care service feedback.

4. The method for determining emotional quotient of claim 3, wherein the evaluating further includes querying whether the user agrees on the recognized emotional state when self diagnosis is used.

5. The method for determining emotional quotient of claim 4, wherein the re-determining includes:
if agreed on the recognized emotional state by the response to the query, fixing the emotional variation threshold values in an emotional threshold management table, and
if not, adjusting the level of the information sensed from the bio signals and the environmental signals to correct the emotional variation threshold value.

6. The method for determining emotional quotient of claim 3, wherein the evaluating further includes:
analyzing service consuming patterns according to the user's emotional state to select the emotional care service meeting the emotional quotient when the emotional care service feedback is used;
monitoring the emotional variation while the selected emotional care service is provided; and
analyzing the emotional variation in the current emotional quotient according to the monitored results.

7. The method for determining emotional quotient of claim 6, wherein the re-determining further includes:
when the current emotion of the user is not varied, adjusting the level of the emotional quotient if it is determined that it is negative emotion by searching the emotional quotient, and
if not, fixing the emotional variation threshold values in the emotional threshold management table.

8. The method for determining emotional quotient of claim 6, wherein the re-determining includes:
when the current emotion of the user is varied, confirming whether the emotion is varied to the positive emotion; and
when the emotion is varied to the positive emotion, fixing the emotional variation threshold values in the emotional threshold management table.

9. The method for determining emotional quotient of claim 8, further comprising when the current emotion of the user is varied, confirming whether the emotion is varied to the negative emotion; and
when the emotion is varied to the negative emotion, adjusting the level of the emotional quotient.

10. An apparatus for determining emotional quotient according to emotional variation, comprising:
a plurality of sensors that sense bio signals and environmental signals;
a sensing signal processor that processes sensing signals sensed by the plurality of sensors; and
an emotional signal processor that defines emotional variation threshold values as a reference capable of recognizing emotional variation, analyzes the sensing signals from the sensing signal processor to extract emotional signal, and infers the emotional quotient based on the extracted emotional signal and the environment signals.

11. The apparatus for determining emotional quotient according to emotional variation of claim 10, wherein the emotional signal processor further includes:
an error condition recognizing unit that analyzes the environmental information to sense the error condition of the extracted emotional signal; and
an emotional signal compensator that compensates the error condition recognized by the error condition recognizing unit to optimize the emotional signal.

12. The apparatus for determining emotional quotient according to emotional variation of claim 11, wherein the emotional signal compensator adjusts the level of the emotional quotient to compensate for the error condition of the emotional signal.

13. The apparatus for determining emotional quotient according to emotional variation of claim 10, wherein the emotional signal processor extracts the emotional signal from the bio signals based on the emotional variation threshold values.

14. The apparatus for determining emotional quotient according to emotional variation of claim 10, wherein the emotional signal processor further includes:
an emotional signal information managing unit that manages the emotional variation threshold values, the emotional quotient, the emotional signal, and the state information of the emotional signal feedback from the corresponding user.

15. The apparatus for determining emotional quotient according to emotional variation of claim 14, wherein the emotional signal information managing unit evaluates the emotional quotient based on the feedback result from the user and determines the optimized emotional variation threshold values and the emotional quotient according to the evaluation results.

16. The apparatus for determining emotional quotient according to emotional variation of claim 14, wherein the emotional signal information managing unit evaluates the emotional quotient through the self diagnosis or the emotional care service feedback for the emotional state recognition result.

17. The apparatus for determining emotional quotient according to emotional variation of claim 16, wherein the emotional signal information managing unit queries whether the user agrees on the recognized emotional state when the emotional quotient is evaluated through the self diagnosis, to evaluate the evaluation quotient according to the response from the user.

18. The apparatus for determining emotional quotient according to emotional variation of claim 16, wherein the emotional signal information managing unit monitors the emotional variation while the emotional care service is provided when the emotional quotient is evaluated through the emotional care service feedback, to confirm whether the emotion for the recognized emotional state is varied and to evaluate the emotional quotient according to the result.
